(19) United States
(12) Patent Application Publication
(10) Pub. No.: US 2022/0185882 A1
(43) Pub. Date: Jun. 16, 2022

(54) ARTIFICIAL IMMUNOSURVEILLANCE CHIMERIC ANTIGEN RECEPTOR (AI-CAR) AND CELLS EXPRESSING THE SAME

(71) Applicant: ARBELE Limited, Shatin, N.T. (HK)

(72) Inventors: Donald E. Staunton, Kirkland, WA (US); John M. Luk, BELLEVUE, WA (US); John M. Harlan, Seattle, WA (US)

(21) Appl. No.: 17/432,922

(22) PCT Filed: Feb. 21, 2020
(86) PCT No.: PCT/US2020/019376

§ 371 Date: Aug. 21, 2021

Related U.S. Application Data
(60) Provisional application No. 62/808,815, filed on Feb. 21, 2019.
(60) Provisional application No. 62/808,823, filed on Feb. 21, 2019.
(60) Provisional application No. 62/808,833, filed on Feb. 21, 2019.

(51) Int. Cl. C07K 16/28; C07K 14/725; C07K 14/705; C07K 14/715; C07K 16/32; C07K 16/30; C07K 14/54; C07K 14/52

(52) U.S. Cl. CPC C07K 16/2803; C07K 14/7051; C07K 14/70517; C07K 14/7155; C07K 16/32; C07K 2319/03; C07K 16/2818; C07K 14/5418; C07K 14/523; C07K 2317/622; C07K 16/30

(57) ABSTRACT

The application provides non-viral vector, comprising an artificial immunosurveillance chimeric antigen receptor (AI-CAR) expression cassette flanked by two transposons or viral terminal repeats (IR), wherein the AI-CAR expression cassette comprises an inducible gene expression unit and a CAR expression unit.

### CROSS REFERENCE TO RELATED APPLICATIONS

This application is a national entry from PCT/US2020/019376 and claims the benefit of the filing date of U.S. Provisional Application Ser. No. 62/808,815 filed Feb. 21, 2019, U.S. Provisional Application Ser. No. 62/808,823 filed Feb. 21, 2019, and U.S. Provisional Application Ser. No. 62/808,833, filed Feb. 21, 2019 under 35 U.S.C. 119(e), the entire disclosures of which are incorporated by reference herein.

### TECHNICAL FIELD

This invention relates to the technology for improving the expansion, manufacturing, survival and efficacy of chimeric antigen receptor (CAR)-T cells or NK cells.

### BACKGROUND

Unless otherwise indicated herein, the materials described in this section are not prior art to the claims in this application and are not admitted to be prior art by inclusion in this section.

Manufacturing CAR cells is laborious and can be complicated by the need for artificial antigen presenting cells (aAPC), antibody stimulation of TCR, and co-stimulatory receptors and/or multiple cytokines to expand autologous or allogenic CAR cells prior to administration. For example, electroporation of T cells, NK cells, PBLs or PBMC with DNA vectors of CAR typically results in cell death of the majority of cells when the electroporation conditions are set for high percent CAR expression. Thus, following electroporation, the cells may be co-cultured with irradiated aAPCs, antibodies, and/or growth factors, and the CAR cell population may be specifically expanded multiple folds in order to produce a single dose for therapeutic use. Currently, the standard CAR vectors (CARS) do not express genes capable of inducing a patient's anti-tumor immune response, which is often needed in an immunosuppressed tumor microenvironment.

Ideally, the efficacy of CAR cells following administration should correlate with the T cells having an undifferentiated memory phenotype, characterized by the in vivo persistence and the greatest therapeutic potential. To selectively expand the CAR cells with this phenotype and/or prevent terminal differentiation, several cytokines have been utilized, including IL15, IL7 and IL21. IL15 and IL7 are known to be critical for generating and supporting early memory T cells due to their ability of instructing the generation of human memory stem T cells from naive precursors (Cieri et al., 2013; Boyman et al., 2012; Gattinoni L, et al., 2011). IL15 and IL7 may be instrumental for de-differentiating the T cells, such as human CD8+ memory T-cell subsets in response to antigen or homeostatic cytokines (Geginat 2003). IL15 is required for innate-like T cell immunosurveillance (Dadi S, et al., 2016). The soluble and transpresented IL15/IL-15Ralpha enables sustained IL-15 activity and contributes to the long survival of CD8 memory T cells (Sato, et al., 2007). Therefore, CAR-T cells with an undifferentiated memory phenotype demonstrate the greatest in vivo persistence and therapeutic efficacy.

However, all of the components used to manufacture and maintain such CAR cells with high therapeutic potential cannot be co-administered due to the safety assessment for each component. Yet, building these components into the CAR vector with constitutive expression may not be a viable practice because of the high probability of toxicity overtime. Alternatively, if the signaling pathways of CAR cell expansion and undifferentiated memory phenotype are under the control of a single molecule, for example, a target tumor antigen, it would enable not only a great reduction in cost but also a point of care treatment. In this way, the CAR signaling may be sufficient to support the expansion of CAR cells without terminal differentiation.

From the clinical point of view, multiple factors contribute to the relapse of treatment, including insufficient CAR cell persistence (exhaustion or host anti-CAR), loss of target antigen, lack of inducing a host anti-tumor response, and/or inability to efficiently locate to lymphoma/solid tumors. Almost all forms of CAR address loss of tumor antigen addressed by targeting 2 or more tumor antigens. Thus, it is highly desirable to have one or more CARs together with a durable host response by inclusion of additional genes into the CAR vector/RNA.

### SUMMARY

In one aspect, the application provides chimeric antigen receptor complex. In one embodiment, the chimeric antigen receptor complex comprises a first protein, comprising a first extracellular domain linked to a first intercellular domain through a first linker, wherein the first extracellular domain comprises a first scFv having affinity towards a first tumor epitope, and wherein the first intercellular domain comprises a JAK1 binding domain, and a second protein, comprising a second extracellular domain linked to a second intercellular domain through a second linker, wherein the second extracellular domain comprises a second scFv having affinity towards a second tumor epitope, and wherein the second intercellular domain comprises a JAK3 binding domain. The first tumor epitope is on a first tumor antigen. The second tumor epitope is on a second tumor antigen.

In one embodiment, the first intracellular domain comprises IL7Rα(CD127). In one embodiment, the first intracellular domain comprises intracellular domain of IL15Rβ(CD122), IL21Rα(CD360), or a combination thereof. In one embodiment, the first intracellular further comprises a first cytotoxic signaling domain linked to a JAK1 binding domain.

In one embodiment, the first cytotoxic signaling domain comprises CD28, CD3ζ, CD137, OX40, CD27, ICOS, or a combination thereof.

In one embodiment, the first scFv domain or the second scFv domain independently has an affinity toward CD19 or CD22. In one embodiment, the first scFv domain has an affinity toward CD19. In one embodiment, the second scFV domain has an affinity toward CD22. In one embodiment, the second intracellular domain comprises γ(CD132).

In one embodiment, the second intracellular domain further comprises a second cytotoxic signaling domain linked to a JAK3 binding domain. In one embodiment, the second cytotoxic domain comprises CD28, CD3 [symbol] , CD137, OX40, CD27, ICOS, or a combination thereof. In one embodiment, the second intracellular domain comprises in tandem γ(CD132), JAK3 binding domain, CD28, and CD3ζ. In one embodiment, the first intracellular domain is configured to dimerize with the second intracellular domain.

In one embodiment, the first and the second linker comprises independently CD8. In one embodiment, the first and the second linker comprises independently a stalk and a transmembrane domain.

In one embodiment, the stalk comprises CD8, Fc hinge, Fc CH2-CH3, TCRα, TCRβ, truncated IL7Rα(CD127), truncated IL15Rβ(CD122), IL15Rα(CD215), truncatedγ (CD132), truncated IL21Rα(CD360), or a combination thereof.

In one embodiment, the transmembrane domain comprises CD8, CD28, CD3ζ, CD3ε, CD3δ, CD3γ, CD3ζ, TCRα, TCRβ, IL15Rβ (CD122), γ(CD132), IL7Rα (CD127), IL21Rα (CD360), IL15Rα (CD215), or a combination of.

In one embodiment, the tumor antigen comprises CDH17, TROP2, CD19, CD22, CD37, BCMA, CD48, EGFR, HER2, EpCAM, CEACAM5, PSMA, GD2, GPC3, or a combination of.

In another aspect, the application provides open reading frames (ORFS). In one embodiment, the open reading frame (ORF) comprises sequentially CD19 scFv, a stalk trans-membrane region, and an IL7 alpha endo-domian. In one embodiment, the open reading frame (ORF) comprises sequentially CD22 scFv, a stalk trans-membrane region, a gamma chain endo-domain, CD28 endo-domain, and CD3 [symbol] endo-domain. In one embodiment, the open reading frame (ORF), comprising sequentially PD-1 scFv, CCL21, and IL7.

In a further aspect, the application provides biomolecule complexes. In one embodiment, the biomolecule complexes comprises a first protein, comprising a first extracellular domain linked to a first intercellular domain through a first linker, wherein the first extracellular domain comprises a first scFv having affinity towards a first tumor epitope, and wherein the first intercellular domain comprises a JAK1 binding domain, a second protein, comprising a second extracellular domain linked to a second intercellular domain through a second linker, wherein the second extracellular domain comprises a second scFv having affinity towards a second tumor epitope, and wherein the second intercellular domain comprises JAK3 domain, a first tumor antigen, and a second tumor antigen. The first tumor epitope is bound a first tumor antigen. The second tumor epitope is bound to the tumor antigen.

In one embodiment, the first intracellular domain is dimerized with the second intracellular domain. In one embodiment, JAK1 is dimerized with JAK3.

In a further aspect, the application provides non-viral DNA constructs. In one embodiment, the non-viral DNA construct comprises sequentially from 5′ to 3′, an inducible promotor followed by a first ORF, wherein the first ORF comprises anti-PD-1 scFV, CLL21 and IL7, each lead with a single peptide and end with a ribosomal skipping peptide, a second ORF comprising at least one constitutive chimeric antigen receptor, and a third promotor followed by at least one RNA sequence.

In a further aspect, the application provides chimeric antigen receptors. In one embodiment, the chimeric antigen receptor comprises sequentially, a cytokine domain, a linker, a truncated CD8 domain, and a signaling endo-domain.

In one embodiment, the cytokine domain comprises IL7, IL12, IL21, or a combination thereof. In one embodiment, the truncated CD8 domain comprises a hinge, a transmembrane domain, and at least a portion of a cytoplasmic domain. In one embodiment, the cytoplasmic domain comprises CD28/CD170, CD3ζ, or a combination thereof.

In one embodiment, chimeric antigen receptor further comprises a tumor antigen domain intermediating the cytokine domain and the truncated CD8 domain.

In one embodiment, the application provides biomolecule complexes, comprising the chimeric antigen receptors as disclosed thereof bound with a tumor antigen.

In a further aspect, the application provides non-viral vector, comprising an artificial immunosurveillance chimeric antigen receptor (AI-CAR) expression cassette flanked by two transposons or viral terminal repeats (IR), wherein the AI-CAR expression cassette comprises an inducible gene expression unit and a CAR expression unit.

In one embodiment, the inducible gene expression unit comprises a STAT, NFAT, or NF-kB inducible promoter, a coding region for one or more genes linked by an IRES or a self-cleaving ribosomal skip peptide, followed by a first polyA signal sequence. In one embodiment, the self-cleaving ribosomal skip peptide comprises TA2. In one embodiment, the inducible gene expression unit comprises genes for expressing at least two different cytokine receptors. In one embodiment, the inducible gene expression unit comprises genes for expressing an antigen binding protein. In one embodiment, the inducible gene expression unit comprises genes for expressing anti-PD1 scFv. In one embodiment, the inducible gene expression unit comprises genes for expressing CCL21. In one embodiment, the inducible gene expression unit comprises genes for expressing IL7.

In one embodiment, the CAR expression unit comprises genes for expressing anti-CDH17 scFv, anti-TROP2 scFv, and CAR. In one embodiment, the CAR expression unit comprises a promoter, one or two CAR genes, followed by a second polyA signal sequence. In one embodiment, the CAR expression unit further comprises a gene for expressing a safety switch. In one embodiment, the safety switch comprises a truncated EGFR (tEGFR) or truncated CD20. In one embodiment, the AI-CAR expression cassette is configured to express shRNA, wherein the shRNA is configured to inhibit the endogenous TCR.

In a further aspect, the application provides isolated nucleic acids, encoding the biomolecule complexes, biomolecules, antigens, and proteins as disclosed thereof.

In a further aspect, the application provides expression vectors, comprising the isolated nucleic acids as disclosed thereof. In one embodiment, the expression vector comprises the ORFs as disclosed thereof. In one embodiment, the expression vector comprises the non-viral DNA constructs as disclosed thereof. The expression vectors may be viral or non-viral. The vector may be expressible in a cell.

In a further embodiment, the application provides host cells. In one embodiment, the host cell comprises the isolated nucleic acids and/or the expression vectors as disclosed thereof. In one embodiment, the host cell comprises the non-viral DNA construct as disclosed thereof. In one embodiment, the host cell comprises the non-viral vectors as disclosed thereof.

In a further embodiment, the application provides mammalian cells, comprising the chimeric antigen receptor complex, the biomolecule complexes, the biomolecules, the antigens, and proteins as disclosed thereof. In one embodiment, the mammalian cell comprises the chimeric antigen receptor as disclosed thereof. In one embodiment, the mammalian cell comprises the biomolecule complex as disclosed thereof.

In a further embodiment, the application provides CAR-T or CAR-NK cells. In one embodiment, the CAR-T or CAR-NK cells express the chimeric antigen receptor complexes as disclosed thereof. In one embodiment, the CAR-T or CAR-NK cell expresses the chimeric antigen receptor as disclosed thereof.

In a further embodiment, the application provides methods for treating tumor in a subject, comprising administering to the subject a sufficient amount of the CAR-T or CAR-NK cell as disclosed thereof.

In a further aspect, the application provides pharmaceutical compositions. In one embodiment, the pharmaceutical composition comprises a therapeutically effective amount of the vectors, non-viral vectors, CAR-T or CAR-NK cell, proteins, biomolecules, or biomolecule complexes as disclosed thereof. In one embodiment, the pharmaceutical composition further comprises a pharmaceutically acceptable vehicle.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 depicts AI-CAR gene expression cassette comprising an inducible gene expression unit and the CAR expression unit in a non-viral vector (pPI) for constitutively expressed one or two CARs to induce gene expression for a host anti-tumor response;

FIG. 2 displays a general concept of AI-CARs;

FIG. 3 depicts an AI-CAR expression vector encodes constitutively expressed dual CARs that target CDH17 and TROP2 and a cassette of anti-PD1 scFv, CCL21, and IL17 genes under an inducible promoter in a non-viral vector (pPI) for a CAR-induced host anti-tumor response; An AI-CAR expression vector encodes constitutively expressed dual CARs that target CDH17 and TROP2 and a cassette of anti-PD1 scFv, CCL21, and IL17 genes under an inducible promoter in a non-viral vector (pPI) for a CAR-induced host anti-tumor response;

FIG. 4 shows tumor antigen induction of integrated pPI anti-CDH17 AI-CAR vector gene; Tumor antigen induction of integrated pPI-anti-CDH17-AI-CAR vector gene. (A) The expression of integrated pPI-anti-CDH17-AI-CAR vector was measured by the level GFP in T cells (Jurkat) in response to different concentrations of CDH17; (B) Induction with recombinant CDH17 in colon cancer cells (SW480); and (C) cytotoxicity of pPI-anti-CDH17-AI-CAR integrated T cells to SW480 cells expressing CDH17;

FIG. 5 shows the expression and binding specificity of a pPI-anti-CDH17-TROP2 AI-CAR; and

FIG. 6 depicts variants of iPro to support proliferation and persistence of AI-CAR. Variants of iPro to support AI-CAR proliferation and persistence. (A) An example of iPro7 expression; (B) induction of proliferation of CD25 T cell population; and (C) increased T cell survival.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The disclosure provides, among others, isolated antibodies, methods of making such antibodies, bispecific or multi-specific molecules, antibody-drug conjugates and/or immuno-conjugates composed from such antibodies or antigen binding fragments, pharmaceutical compositions containing the antibodies, bispecific or multi-specific molecules, antibody-drug conjugates and/or immuno-conjugates, the methods for making the molecules and compositions, and the methods for treating cancer using the molecules and compositions disclosed herein.

The term “antibody” is used in the broadest sense and specifically covers single monoclonal antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, single chain, bispecific or bi-effective, simianized, human and humanized antibodies as well as active fragments thereof. Examples of active fragments of molecules that bind to known antigens include Fab, F(ab′)₂, scFv and Fv fragments, including the products of an Fab immunoglobulin expression library and epitope-binding fragments of any of the antibodies and fragments mentioned above. In some embodiments, antibody may include immunoglobulin molecules and immunologically active portions of immunoglobulin molecules, i.e. molecules that contain a binding site that immunospecifically bind an antigen. The immunoglobulin can be of any type (IgG, IgM, IgD, IgE, IgA and IgY) or class (IgG1, IgG2, IgG3, IgG4, IgA1 and IgA2) or subclasses of immunoglobulin molecule. In one embodiment, the antibody may be whole antibodies and any antigen-binding fragment derived from the whole antibodies. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler & Milstein, Nature, 256:495 (1975), or may be made by recombinant DNA methods (see, e.g., U.S. Pat. No. 4,816,567).

The monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

Monoclonal antibodies can be produced using various methods including mouse hybridoma or phage display (see Siegel. Transfus. Clin. Biol. 9:15-22 (2002) for a review) or from molecular cloning of antibodies directly from primary B cells (see Tiller. New Biotechnol. 28:453-7 (2011)). In the present disclosure antibodies were created by the immunization of rabbits with both human PD-L1 protein and cells transiently expressing human PD-L1 on the cell surface. Rabbits are known to create antibodies of high affinity, diversity and specificity (Weber et al. Exp. Mol. Med. 49:e305). B cells from immunized animals were cultured in vitro and screened for the production of anti-PD-L1 antibodies. The antibody variable genes were isolated using recombinant DNA techniques and the resulting antibodies were expressed recombinantly and further screened for desired features such as ability to inhibit the binding of PD-L1 to PD-1, the ability to bind to non-human primate PD-L1 and the ability to enhance human T-cell activation. This general method of antibody discovery is similar to that described in Seeber et al. PLOS One. 9:e86184 (2014).

The term “antigen- or epitope-binding portion or fragment” refers to fragments of an antibody that are capable of binding to an antigen (CD19 in this case). These fragments may be capable of the antigen-binding function and additional functions of the intact antibody. Examples of binding fragments include, but are not limited to a single-chain Fv fragment (scFv) consisting of the VL and VH domains of a single arm of an antibody connected in a single polypeptide chain by a synthetic linker or a Fab fragment which is a monovalent fragment consisting of the VL, constant light (CL), VH and constant heavy 1 (CH1) domains. Antibody fragments can be even smaller sub-fragments and can consist of domains as small as a single CDR domain, in particular the CDR3 regions from either the VL and/or VH domains (for example see Beiboer et al., J. Mol. Biol. 296:833-49 (2000)). Antibody fragments are produced using conventional methods known to those skilled in the art. The antibody fragments are can be screened for utility using the same techniques employed with intact antibodies.

The “antigen-or epitope-binding fragments” can be derived from an antibody of the present disclosure by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂fragment that has two antigen combining sites and is still capable of cross-linking antigen.

The Fab fragment may contain the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other, chemical couplings of antibody fragments are also known.

“Fv” is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The “light chains” of antibodies (immunoglobulins) from any vertebrate species can be assigned to one of two clearly distinct types, called kappa and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called α, delta, epsilon, γ, and μ, respectively. The subunit structures and three-dimensional configurations of different classes of immunoglobulins are well known.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art. (see, e.g., Queen et al., Proc. Natl Acad Sci USA, 86:10029-10032 (1989), Hodgson et al., Bio/Technology, 9:421 (1991)).

The terms “polypeptide”, “peptide”, and “protein”, as used herein, are interchangeable and are defined to mean a biomolecule composed of amino acids linked by a peptide bond.

The terms “a”, “an” and “the” as used herein are defined to mean “one or more” and include the plural unless the context is inappropriate.

By “isolated” is meant a biological molecule free from at least some of the components with which it naturally occurs. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic a binding specificity.

“Recombinant” means the antibodies are generated using recombinant nucleic acid techniques in exogeneous host cells.

The term “antigen” refers to an entity or fragment thereof which can induce an immune response in an organism, particularly an animal, more particularly a mammal including a human. The term includes immunogens and regions thereof responsible for antigenicity or antigenic determinants.

Also, as used herein, the term “immunogenic” refers to substances which elicit or enhance the production of antibodies, T-cells or other reactive immune cells directed against an immunogenic agent and contribute to an immune response in humans or animals. An immune response occurs when an individual produces sufficient antibodies, T-cells and other reactive immune cells against administered immunogenic compositions of the present disclosure to moderate or alleviate the disorder to be treated.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20−, 50−, 100−, 500−, 1000−, 5,000−, 10,000− or more times greater for a control molecule relative to the antigen or epitope.

Also, specific binding for a particular antigen or an epitope can be exhibited, for example, by an antibody having a KA or Ka for an antigen or epitope of at least 20−, 50−, 100−, 500−, 1000−, 5,000−, 10,000− or more times greater for the epitope relative to a control, where KA or Ka refers to an association rate of a particular antibody-antigen interaction.

“Homology” between two sequences is determined by sequence identity. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs. The deviations appearing in the comparison between a given sequence and the above-described sequences of the disclosure may be caused for instance by addition, deletion, substitution, insertion or recombination.

The present disclosure may be understood more readily by reference to the following detailed description of specific embodiments and examples included herein. Although the present disclosure has been described with reference to specific details of certain embodiments thereof, it is not intended that such details should be regarded as limitations upon the scope of the disclosure.

In a previous disclosure, CAR exhaustion was addressed by cytokine signaling pathways to drive expansion without terminal differentiation. Herein the disclosed composition and method of use is related to the AI-CAR vectors for Artificial Immunesurveillance Chimeric Antigen Receptor. The advancement of this AI-CAR technology aims to replace standard CAR manufacturing and enable an effective ‘combination’ and point of care therapy. While other CAR technologies may require the expression of soluble cytokine growth factors and/or multiple dosing for persistent activity to mount a complete and durable response, AI-CAR vectors incorporates these activities and enables the production of effective and persistent CAR cells in the absence of either a constitutively active driver for proliferation or multiple CAR dosing for a durable anti-tumor response.

Second, while retaining the same function as a standard CAR, AI-CAR signaling increases the efficiency of manufacturing CAR cells. AI-CAR may only require one target antigen for full proliferation and cytotoxic activities both in vitro and in vivo. In this context, AI-CAR may enable substantial reduction in manufacturing costs since the expansion of standard CAR-T cells generally requires the use of a combination of growth factors and aAPC for manufacturing.

Third, following AI-CAR engagement of tumor cells in vivo, the expression of several anti-tumor genes that are encoded by the integrated AI-CAR vector may be induced. The expression of these endogenous genes may enable patients to mount an anti-tumor response that more broadly targets different tumor antigens, such as neoantigens. For example, a STAT5 reporter system is used to induce STAT5 responsive genes in human T cells (Kanai, et al., 2014; Zeng, et al., 2016; Bednorz et al., 2011; and Fang et al., 2008). This feature is unique because standard CAR constructs typically are not capable of inducible gene expression. Together with these co-factors, AI-CAR will become a platform technology providing practical, economic, and effective solutions for the point of care cancer treatment. Many forms of cancer may exist in a tumor environment that is immunosuppressive. AI-CAR will be highly desirable because AI-CAR vectors are designed to express additional anti-cancer genes that can decrease tumor immunosuppression and activate the patient's anti-tumor immune response. One of the unique features of AI-CAR is its ability to regulate the expression of relevant anti-tumor genes at a tumor site and not to have them constitutively expressed which may be toxic. Another characteristic feature is that AI-CAR is designed to have a single dose at administration followed by its long-term activity and greater efficacy. With these advantageous features, AI-CAR is a better solution for the unmet challenge in the market, which promises the efficacy for treating most if not all types of cancer.

As to CAR therapeutics for treating hematologic cancers, targets include CD19, and CD22, CD20, CD9, CD38 that may be targeted by dual, bispecific, AI-CAR that use non-viral DNA vectors or RNA-CAR (transient). In this way, AI-CAR may be used as a transient treatment bridge to transplant or for greater persistence. Moreover, AI-CAR can be used as a point a care treatment to induce a host anti-tumor immune response targeting neoantigens.

In addition, for certain solid tumors, RNA encoding an Al-CAR combination therapy may be applicable. For aggressive tumors that have failed other treatments, off-the-shelf RNA CARs can be efficiently and rapidly manufactured due to the high electroporation efficiency of RNA. The multiple anti-tumor mechanisms transiently expressed by the RNA AI-CAR, like induced AI-CAR vector genes, should enable more effective and safer anti-tumor activity and potentially induce a patient immune response. Multidosing an RNA AI-CAR may serve as a bridge to determine efficacy prior to treatment with a certain, persistent AI-CAR cell.

### EXAMPLES

### Example 1. Construction of AI-CAR Vectors for Persistent and Combination Therapy

The purpose of AI-CAR cells is to improve the efficacy of cancer immune therapy by enabling persistent, long term immunosurveillance in quiescent state until stimulated by tumor cells. Post-tumor stimulation, the inducible AI-CAR genes enable localized safe and more effective ‘combination’ therapy involving additional mechanisms of anti-tumor activity. Stimulation of a patient's anti-tumor response is anticipated for a greater frequency of complete and durable responses.

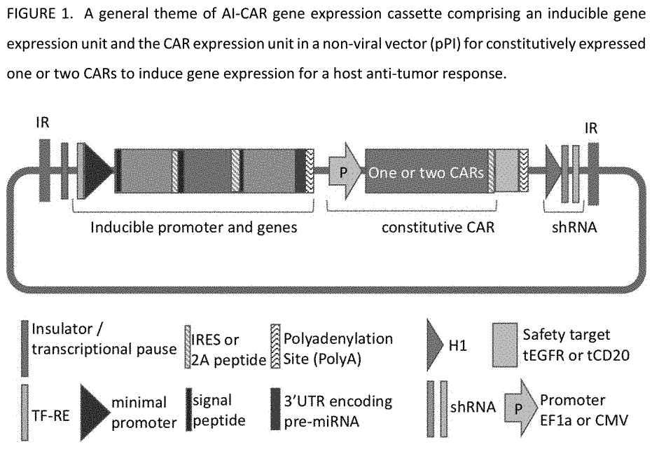

In general, an AI-CAR construct is comprised of an AI-CAR expression cassette in a non-viral vector (pPI), such as transposon-based integration systems (Ivics and Izsvák, 2010; Z Cooper et al., U.S. Pat. No. 9,629,877; Uckert et al., US20190071484A1). As shown in FIG. 1, an AI-CAR vector is comprised of an inducible gene expression unit and the CAR expression unit, i.e. an AI-CAR expression cassettes flanked by transposon terminal inverted repeats (IR). An AI-CAR expression cassette may be constructed with a STAT, NFAT or NF-κB inducible promoter, the coding region for one or more genes linked by an IRES or self-cleaving ribosomal skip peptide, such as P2A or T2A (for example SEQ ID 18-21), and followed by a polyA signal sequence. This may be followed by another promoter for constitutively expressing one or two CARs, which is followed by another polyA signal. For examples of pairs of AI-CAR chains see SEQ ID 1 and 2, 3 and 4, 5 and 6, 7 and 8, 9 and 10. Both coding regions may be located between two transposons or viral terminal repeats (IR) for integration. Alternatively, the coding regions of an AI-CAR construct may be integrated at a specific genomic site using zinc finger, TALEN or CRISPR/Cas9 nucleases (Eyquem 2017).

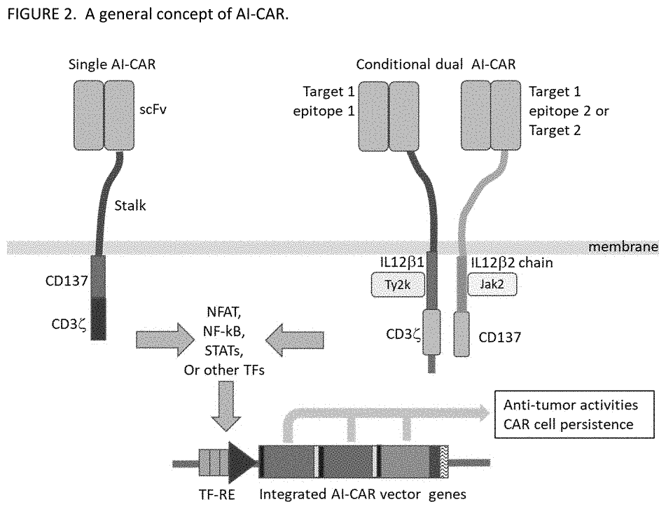

As elucidated in FIG. 2, the constitutive expression of one or two CARs on the surface of T cells binds to tumor associated antigen(s) at a tumor. The AI-CAR induces expression of inducible genes, enabling safe and multiple mechanisms of anti-tumor activity and potent stimulation of a host anti-tumor response. Ideally the expression of these genes will be induced subsequent to AI-CAR engagement with tumor antigens or antigens within a tumor microenvironment (TME). To extend the pool of such genes, a second AI-CAR chain may be expressed from a second vector.

There are at least a few flexibilities in the composition of an AI-CAR non-viral vector, such as piggyBac, Toll, and Sleeping Beauty (Ivics and Izsvák, 2010). Additionally, AI-CAR may express a safety switch, such as a truncated EGFR (tEGFR) which can be targeted for elimination by an FDA-approved antibody, Cetuximab. Alternatively, the safety target may be a truncated CD20 that can be targeted for CAR cell elimination by Rituximab. An AI-CAR vector may express shRNA that for example inhibit the endogenous TCR to enable the generation of universal AI-CAR cells.

As an alternative to an inducible promoter, AI-CAR may include a weak promoter, such as a modified PGK promoter, to safey express the other anti-tumor mechanisms of activity.

### Example 2. Single and Dual AI-CAR Designs

A single or dual Al-CAR is designed to signal through cytokine receptor pathways for greater CAR cell persistence and induce vector encoded genes for additional anti-tumor mechanisms and enhanced efficacy. Moreover, simple engagement of an AI-CAR may facilitate efficient AI-CAR expansion and may be used in manufacturing to simplify in vitro expansion prior to administration.

A dual AI-CAR is constructed with IL12, IL7, IL21 or IL15 cytokine receptor endodomains to enable persistence and induction of vector genes for additional anti-tumor mechanisms. As listed in Table 2, the cytokine endo-domains are fused to one or more TCR or TCR co-stimulatory cytoplasmic region (also known as co-stimulatory domain), such as CD3ζ, CD28, CD137, CD27, OX40 and ICOS. For example, a dual AI-CAR may be constructed with one CAR composed of a tumor antigen specific scFv fused to a stalk and transmembrane domain and a segment of the intracellular (endo) domain of IL12 beta1 chain and CD3ζ. The second CAR may be composed of a tumor antigen specific scFv fused to a stalk and transmembrane domain, a segment of the common beta2 chain endodomain, and a CD137 co-stimulatory endodomain.

AI-CAR may induce gene expression through a transcription factor such as STAT4. The IL12 betal chain possesses a Ty2k binding site whereas the beta2 chain possesses a JAK2 binding site. The association of the two chains may be stabilized by binding to proximal target antigens or to two distinct but proximal epitopes of the same tumor target antigen. Upon CAR engagement with tumor antigens Ty2k and JAK2 will phosphorylate the beta chains and ultimately phosphorylate STAT4 which then dimerizes and translocates to the nucleus. STAT4 may then bind to promoter transcription factor (TF) response elements and promote induction of gene expression. A promoter and downstream genes regulatable by STAT4 may be incorporated into the CAR transposon or viral vectors. Once integrated these genes may be induced subsequent to CAR cells engaging tumor or TME antigens. Endogenous genes induced by STAT4 also support CAR cell persistence (DeRenzo 2019).

Most of dual AI-CAR components are independent functional units, namely, anti-tumor scFv, stalk, transmembrane domain, and endo-domains, as well as different segments of IL15, IL7 and IL21 chains, CD3ζ and co-stimulatory proteins, and subjected to replacement for any number of specific purposes. As examples shown in Table 1 and Table 2, there could be many combinations for manufacturing a specific AI-CAR.

A monospecific AI-CAR may be constructed such that CAR cell persistence is supported by an inducible gene encoded within the CAR vector. The AI-CAR may be composed of an scFv targeting a tumor or TME associated antigen, a stalk, a transmembrane domain and costimulatory CD137 and CD3ζ endodomains. Following engagement of a tumor antigen active NFAT will be generated that binds to response elements within the integrated CAR vector inducing the expression of one or more genes that support persistence, such as IL15, IL12 and IL7 as well as additional mechanisms of anti-tumor activity.

Table 2 shows additional genes encoding proteins or miRNA with persistence activity or anti-tumor activity, such as anti-immune check point inhibitors (ICI), OX40 agonist, TLR agonists, cytokines, bispecific antibodies, iPro, chemokines and chemokine receptors that may be placed under the control of an inducible promoter. Alternatively, mRNA encoding these proteins for example SEQ ID18-20, may be co-transfected with the AI-CAR vector for transient expression. Alternately, mRNA encoding CARs and these proteins may function as AI-CAR for transient, safe therapy (SEQ ID 11-17). Several doses of AI-CAR transiently expressing these genes may suffice to decrease immunosuppression in a tumor microenvironment and activate a patient's anti-tumor immune response.

### Example 3. An AI-CAR Encoding a Bispecific anti-CDH17 and anti-TROP2 CAR and Inducible Genes for Persistence and an Enhanced anti-tumor Response

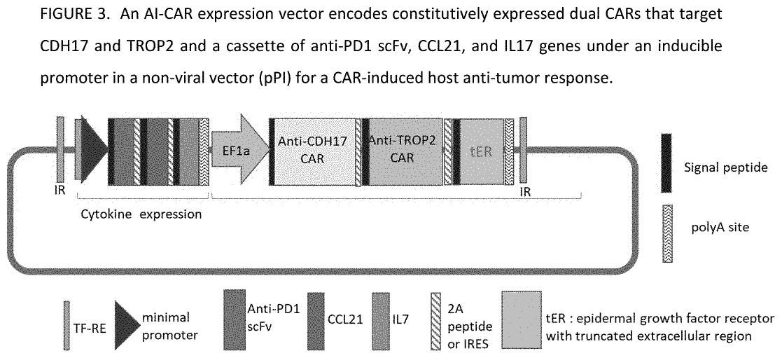

As demonstrated in FIG. 3, an AI-CAR vector may be constructed with an NFAT inducible promoter, the coding region for one or more genes, such as anti-PD-1, CCL21 and IL7, linked by a ribosomal skip peptide, such as T2A, and followed by a polyA signal sequence. This may be followed by a promoter for a single bispecific CAR targeting CDH17 and TROP2 with CD137 and CD3ζ endodomains, followed by T2A, a signal peptide, tEGFR and a polyA signal. Both coding regions may be located between transposon or viral terminal repeats (IR) for integration. Alternatively, the AI-CAR expression cassette may be integrated at a specific genomic site using TALEN or CRISPR/Cas9 (Eyquem 2017).

### Example 4. AI-CAR Gene Induction by Tumor Target Antigen

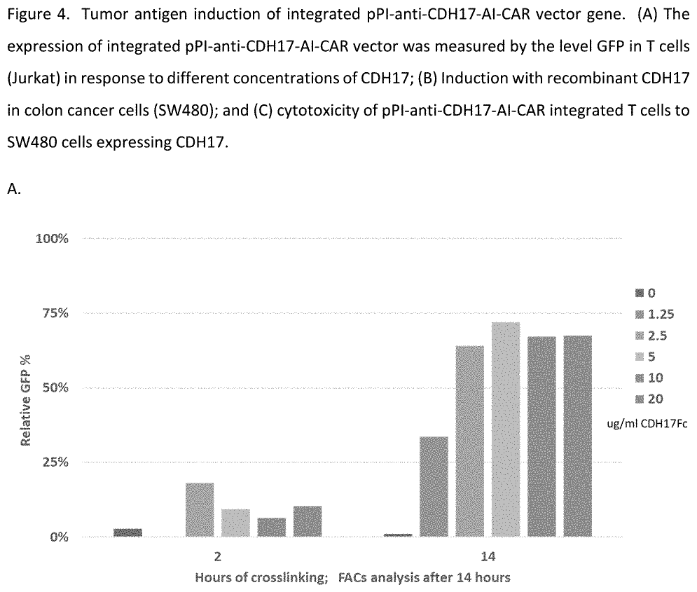

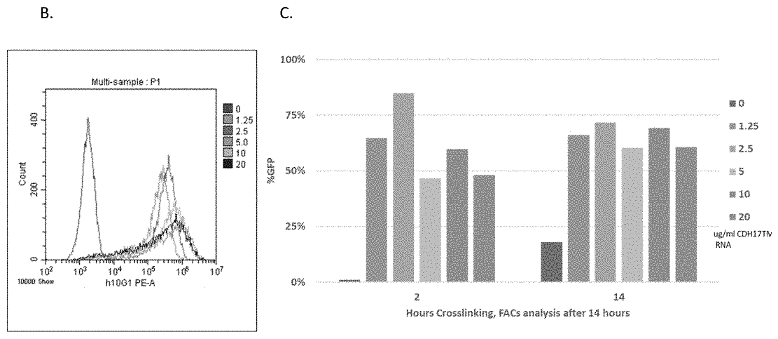

Recombinant and cellular tumor target antigens were used to induced an AI-CAR vector gene following integration into a T cell line. A pPl-anti-CDH17-AI-CAR construct with GFP expression under the control of an NFAT inducible promoter was electroporated into the Jurkat T cell line. This pBac transposon construct was co-electroporated with a transposase expression vector for AI-CAR vector integration. A T cell line (Jurkat) with an integrated pPl anti-CDH17 AI-CAR vector was incubated (37C, 5% CO2) in microtiter wells that were coated with 0, 1.25, 2.5, 5, 10 or 20 ug/ml of CDH17-Fc for 2 hours or 14 hours. The pPl anti-CDH17 AI-CAR vector contains a GFP regulated by an NFAT inducible promoter. At 14 hours the level of GFP expression was determined by flow cytofluorimetry. The level of GFP expression is relative to that maximally induced by 14 hours of lmmunocult treatment (anti- CD3, CD28 and CD2; StemCell). With only 2 hours of CDH17 exposure, low levels of GFP was detected by flow cytometry as shown in FIG. 4A. After 14 hours CDH17 exposure GFP inceased in a concentration dependent manner up to 70% relative to immunocult. There was negiable levels of GFP in unstimulated cells. CDH17 was expressed at different levels in SW480 cells by electroporation with 0, 1.25, 5, 10 or 20 μg of CDH17 RNA (per10\hat{ } 7 cells). Expression of CDH17 was determined by standard flow cytofluorimetry as shown in FIG. 4B. The Jurkat line with integrated pPl anti-CDH17 AI-CAR vector was incubated on a monolayer of SW480 expressing the different levels of CDH17 for 2 or 14 hours. At 14 hours the level of GFP expression was determined by flow cytofluorimetry. The level of GFP expression is relative to that maximally induced by 14 hours of lmmunocult. Similar levels of GFP induction (50-80%) was detected after 2 or 14 hours of AI-CAR exposure to CDH17 expressing Sw480 as shown in FIG. 4C. Thus, tumor cells expressing the target antigen appears to efficiently induce the AI-CAR vector gene expression. This AI-CAR construct may therefore be used to express protiens with anti-tumor activity at a tumor site for safe and enhanced tumor killing.

### Example 5. Expression of Dual AI-CARS

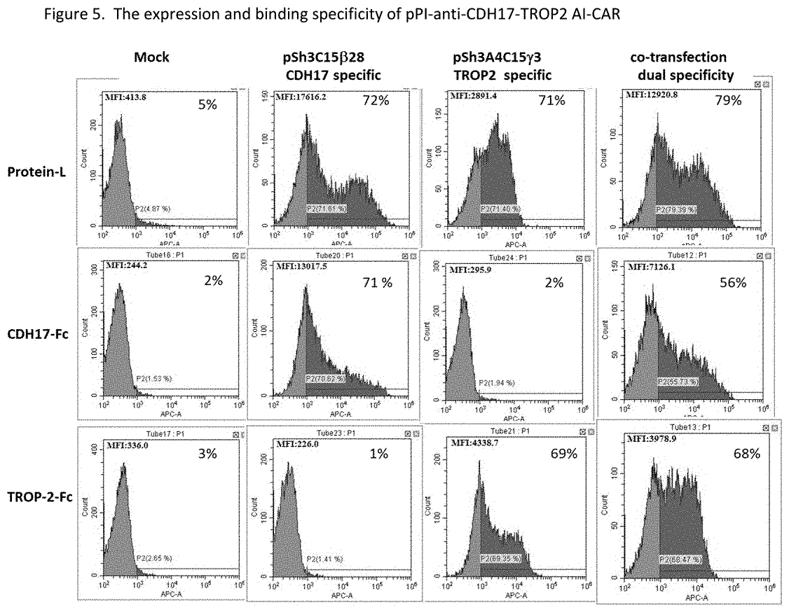

The expression and binding activity of two chain, dual AI-CARS was demonstrated. Dual AI-CARS, one with IL15 beta and CD28 endodomains and the other with IL15 gamma and CD3ζ endodomains(SEQ ID 1 and 2), were expressed in CHO cells. And as illustrated in FIG. 5, the expression of individual chains, pSh3C15b28 and pSh3A4C15g3, or both chains were determined by staining with biotinylated protein-L (for scFv). Binding to tumor antigens, CDH17-Fc or TROP2-Fc was also determined by flow cytofuorimetry. Expression and tumor antigen binding was determined using streptavidin-phycoerythrin for protein-L and anti-human IgG-Alexa647 for CDH17 and TROP2. These results demonstrate that the transfection, expression and ligand binding function of dual CAR can be readily achieved.

### Example 6. Design and Expression of iPro to Support AI-CAR Proliferation and Persistence

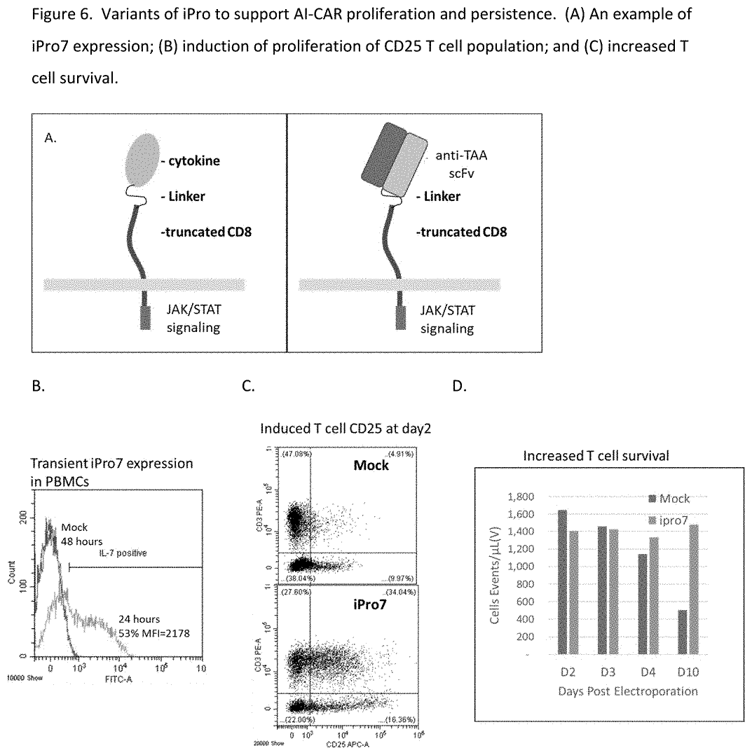

An inducer of proliferation (iPro) may be induced in single AI-CARS to support proliferation and persistence. An iPro may be constructed with a N-terminal cytokine or an scFv that binds a TME antigen, followed by a linker, a stalk, transmembrane domain and an endodomain possessing JAK family and STAT binding sites as shown in FIG. 6A. Following engagement of the N-terminal domain to cytokine receptors or tumor antigens, the iPro may signal through STAT to induce expression of T or NK cell proteins that support survival and maintenance of naïve and Tcm stem phenotype. With a N-terminal cytokine domain, such as IL7, IL15 or IL12. iPro may support bidirectional inside-out and outside-in stimulation that in addition to the AI-CAR cells, activates patient T cells and NK cells. In addition to enhancing in vivo AI-CAR activity, expression of iPro may facilitate the intro expansion of AI-CAR for manufacturing.

An iPro with an N-terminal IL7 (iPro7; SEQ ID 19) transiently expressed in PBMC by electroporation of its in vitro transcribed mRNA is detected at 24 hours but not at 48 hours by flow cytometry (FIG. 6B, left panel). At day 2 induced expression of the IL2 receptor CD25 is demonstrated by flow cytometry (FIG. 6C, middle panel). Without further stimulation T cell counts remain stable over 10 days whereas mock control T cell counts substantially decrease (FIG. 6D, right panel). These results demonstrate that variants of AI-CAR induced genes, such as iPro, may be designed to support AI-CAR proliferation and persistence.

### REFERENCE

Rodriguez-Galán A, Fernandez-Messina L, Sánchez-Madrid F. Control of Immunoregulatory Molecules by miRNAs in T Cell Activation. Front Immunol. 2018 Sep 25;9:2148.
Lykken EA, Li QJ. The MicroRNA miR-191 Supports T Cell Survival Following Common γ Chain Signaling. J Biol Chem. 2016 Nov 4;291(45):23532-23544. Epub 2016 Sep 15.
Ivics, Z., Izsvák, Z. The expanding universe of transposon technologies for gene and cell engineering. Mobile DNA 1, 25 (2010).
Cooper Lj, Torikai H, Zhang L, Huls H, Wang-Johanning F, Hurton L, Olivares S, Krishnamurthy J. Human application of engineered chimeric antigen receptor (CAR) T-cells. US9629877B2.
Uckert W, Bunse M, Clauss J, Izsvák Z. A transposon-based transfection system for primary cells US20190071484A1.

### TABLES

Table 1 lists examples of AI-CAR composition. AI-CARs may be constructed using fragments of different genes encoding the different functional segments including the anti-tumor associated antigen (TAA) scFv, stalk, transmembrane domain, and endo-domains. The different classes of endo-domains may function for example in signaling a cytokine receptor proliferation and survival response or a tumor cytotoxicity response. The cytokine receptor, CD3ζ and co-stimulatory endomains may be fused in various tandem arrangements.

| Functional domain | Target or contributor |
|---|---|
| Anti-tumor associated | CDH17, TROP2, CD19, CD20, CD22, CD37, BCMA, CD48, EGFR, HER2, |
| antigen | EpCAM, CEACAM5, PSMA, GD2, GPC3, FAPa, Claudin18.2 |
| Stalk | CD8, Fc hinge, Fc CH2-CH3, TCR α, TCR β, Truncated IL7Rα (CD127) |
| | Truncated IL15Rβ (CD122), IL15 Rα (CD215), Truncated γ (CD132) |
| | Truncated IL21Rα (CD360) |
| Transmembrane | CD8, CD28, CD3z, CD3ε, CD3δ, CD3γ, CD3ζ, TCRα, TCRβ, |
| | IL15Rβ (CD122), γ (CD132), IL7Rα (CD127), IL21Rα (CD360), |
| | IL15Rα (CD215), IL12Rβ1, (CD212), IL12Rβ2 |
| Cytokine Receptor | IL15Rβ (CD122) or truncated β, γ (CD132) or truncated γ, |
| Signaling | IL7Rα (CD127) or truncated CD127, IL15Rα (CD215) or truncated |
| Endodomains | α, IL21Ra (CD360) or truncated CD360, IL12Rβ1 (CD212), IL12Rβ2 |
| CD3ζ and Co- | None, CD3ζ, CD28, CD137, OX40, CD27, ICOS |
| Stimulatory Signaling | |
| Endodomains | |

Table 2 lists examples of inducible genes that may be incorporated into AI-CAR vectors. These genes may be selected to enhance CAR localization to tumor (e.g. lymphoma), reverse tumor immunosuppression and stimulate host immune response or for direct anti-tumor cell activity. The genes may be placed downstream of a STAT5 inducible promoter to avoid any toxicity that may occur with long term constitutive expression. Alternatively, certain chemokine and chemokine receptor genes and cytokines, e.g. IL7 may be placed downstream of a weak promoter. Low levels of expression may avoid toxicity.

| Inducible genes | Inducible function |
|---|---|
| OX40L (CD134L/CD252) | Dendritic cell activation & maturation. Activates and prolongs |
| agonist | survival of T_{eff} inhibits T_{reg} activity |
| TLR agonists | Activation & maturation of dendritic cells (cDC/mDC), e.g. peptide |
| | or scFv TLR4 agonists TLR4 increases MHC class 1 |
| T or NK cell co- | Agonistic scFv specific for CD137, ICOS, CD40, GITR, A3 adenosine |
| stimulatory membrane | receptor, TNFRs, NTB-A, NKp80, CD48, NKG2 |
| protein agonists | |
| Cytokines, iPro | IL-12, IL7, IL15, IL18, IL21, IFNγ iPro |
| Chemokines and | CCL5, CCL21, CXCL10, CCL19, XCL1, CXCL12, CXCL8, CXCL9, CXCL14 |
| chemokine receptors | CLCL9; CCR2b, CCR3, CCR8, CCR9. Recruitment of CAR and host T |
| | cells, NK cells, and dendritic cells. |
| Checkpoint inhibitor | For example, antibody-based antagonists of CTLA4, PD-1, PD-L1/L2, |
| antagonists | TIGIT, TIM3, LAG3. Reversal of tumor immunosuppression |
| Multispecific antibody | Additional mechanisms of anti-tumor activity |
| | For example; bispecific antibody targeting CDH17 and CD3/NKG2D |
| | or TROP2 and CD3/NKG2D/TRAILR2 |
| miRNA | Decreased AICD, increased CAR cell cytotoxic activity; inhibition of |
| | ICI, miR-191, miR-19b, miR-20b, miR-138, miR-155, miR-181a-Sp, |
| | miR-19, iR-466a-3p |

What is claimed is:

1. A chimeric antigen receptor complex, comprising
a first protein, comprising a first extracellular domain linked to a first intercellular domain through a first linker, wherein the first extracellular domain comprises a first scFv having affinity towards a first tumor epitope, and wherein the first intercellular domain comprises a JAK1 binding domain, and
a second protein, comprising a second extracellular domain linked to a second intercellular domain through a second linker, wherein the second extracellular domain comprises a second scFv having affinity towards a second tumor epitope, and wherein the second intercellular domain comprises a JAK3 binding domain;
wherein the first tumor epitope is on a first tumor antigen, and wherein the second tumor epitope is on a second tumor antigen.

2. (canceled)

3. The chimeric antigen receptor complex of claim 1, wherein the first intracellular domain comprises intracellular domain of IL15Rβ(CD122), IL21Rα (CD360), IL7Rα(CD127), or a combination thereof.

4. The chimeric antigen receptor complex of claim 1, wherein the first intracellular domain further comprises a first cytotoxic signaling domain linked to a JAK1 binding domain.

5. The chimeric antigen receptor complex of claim 4, wherein the first cytotoxic signaling domain comprises CD28, CD3ζ, CD137, OX40, CD27, ICOS, or a combination thereof.

6. (canceled)

7. The chimeric antigen receptor complex of claim 1, wherein the first scFv domain has an affinity toward CD19, and wherein the second scFv domain has an affinity toward CD22.

8-9. (canceled)

10. The chimeric antigen receptor complex of claim 1, wherein the second intracellular domain further comprises a second cytotoxic signaling domain linked to a JAK3 binding domain.

11. The chimeric antigen receptor complex of claim 1, wherein the second cytotoxic domain comprises CD28, CD3 [symbol] , CD137, OX40, CD27, ICOS, or a combination thereof.

12. The chimeric antigen receptor complex of claim 1, wherein the second intracellular domain comprises in tandem γ (CD132), JAK3 binding domain, CD28, and CD3ζ.

13. The chimeric antigen receptor complex of claim 1, wherein the first intracellular domain is configured to dimerize with the second intracellular domain.

14. The chimeric antigen receptor complex of claim 1, wherein the first and the second linker comprises independently CD8.

15. The chimeric antigen receptor complex of claim 1, wherein the first and the second linker comprises independently a stalk and a transmembrane domain.

16. The chimeric antigen receptor complex of claim 13, wherein the stalk comprises CD8, Fc hinge, Fc CH2-CH3, TCRα, TCRβ, truncated IL7Rα (CD127), truncated IL15Rβ (CD122), IL15Rα (CD215), truncatedy (CD132), truncated IL21Rα (CD360), or a combination thereof.

17. The chimeric antigen receptor complex of claim 13, wherein the transmembrane domain comprises CD8, CD28, CD3ζ, CD3ε, CD3δ, CD3γ, CD3ζ, TCRα, TCRβ, IL15Rβ (CD122), γ(CD132), IL7Rα (CD127), IL21Rα (CD360), IL15Rα (CD215), or a combination of.

18. The chimeric antigen receptor complex of claim 1, wherein the tumor antigen comprises CDH17, TROP2, CD19, CD22, CD37, BCMA, CD48, EGFR, HER2, EpCAM, CEACAM5, PSMA, GD2, GPC3, or a combination of.

21-22. (canceled)

23. An open reading frame (ORF), comprising sequentially PD-1 scFv, CCL21, and IL7.

24. A biomolecule complex, comprising
a first protein, comprising a first extracellular domain linked to a first intercellular domain through a first linker, wherein the first extracellular domain comprises a first scFv having affinity towards a first tumor epitope, and wherein the first intercellular domain comprises a JAK1 binding domain,
a second protein, comprising a second extracellular domain linked to a second intercellular domain through a second linker, wherein the second extracellular domain comprises a second scFv having affinity towards a second tumor epitope, and wherein the second intercellular domain comprises JAK3 domain, and
a first tumor antigen, and
a second tumor antigen,
wherein the first tumor epitope is bound a first tumor antigen, wherein the second tumor epitope is bound to the tumor antigen.

25-26. (canceled)

27. A non-viral DNA construct, comprising sequentially from 5′ to 3′,
an inducible promotor followed by a first ORF, wherein the first ORF comprises anti-PD-1 scFv, CLL21 and IL7, each lead with a single peptide and end with a ribosomal skipping peptide,
a second ORF comprising at least one constitutive chimeric antigen receptor, and
a third promotor followed by at least one RNA sequence.

28. A chimeric antigen receptor, comprising sequentially,
a cytokine domain, wherein the cytokine domain comprises IL7, IL12, IL21, or a combination thereof,
a linker,
a truncated CD8 domain, and
a signaling endo-domain.

29-32. (canceled)

33. A non-viral vector, comprising an artificial immunosurveillance chimeric antigen receptor (AI-CAR) expression cassette flanked by two transposons or viral terminal repeats (IR), wherein the AI-CAR expression cassette comprises an inducible gene expression unit and a CAR expression unit.

34. The non-viral vector of claim 33, wherein inducible gene expression unit comprises a STAT, NFAT, or NF-KB inducible promoter, a coding region for one or more genes linked by an IRES or a self-cleaving ribosomal skip peptide, followed by a first polyA signal sequence.

35-45. (canceled)


SEQUENCE LISTING (21 sequences)

SEQ ID NO: 1 - protein, 532 aa, Artificial Sequence
  synthesized
MEFGLSWVFLVALLRGVQCQVQLVQSGAEVKKPGASVKVSCKASGYTFTDFYMNWVRQAPGQGLEWMGRVNPSNGDTNYNQKFKGRVTSTRDTSISTAYMELSRLRSDDTAVYYCARERIYYGISWYFDVWDTGTTVTVSSGGGGSGGGGSGGGGSDIQMTQSPSSLSASVGDRVTITCRASGNIHNYLAWYQQKPGKAPKLLLYNAKTLAEGVPSRFSGSGSGTDYTLTISSLQPEDFATYYCHHYYSTPPTFGQGTKLEIKGAPTTTPAPRPPTPAPTIASQPLSLRPEACRPAAGGAVHTRGLDFACDAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - protein, 496 aa, Artificial Sequence
  synthesized
MEFGLSWVFLVALLRGVQCQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKGAPTTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFRSKRSRGGHSDYMNMTPRRPGPTRKHYQPYAPPRDFAAYRSP

SEQ ID NO: 3 - protein, 530 aa, Artificial Sequence
  synthesized
MKHLWFFLLLVAAPRWVLSQVQLQESGPGLVKPSQTLSLTCTVSGVSLPDYGVSWIRQHPGKGLEWIGVIWGSETTYYNSALKSRVTISVDTSKNQFSLKLSSVTAADTAVYYCARHYYYGGSYAMDYWGQGTLVTVSSGGGGSGGGGSGGGGSDIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGGGTKVEIKGAPTTTPAPRPPTPAPTIASQPLSLRPEASRPAAGGAVHTRGLDFAADAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 4 - protein, 528 aa, Artificial Sequence
  synthesized
MKHLWFFLLLVAAPRWVLSQVQLQESGPGLVRPSQTLSLTCTVSGFTFTSYGVHWVRQPPGRGLEWIGVMWRGGSTDYNAAFMSRVTMLVDTSKNQFSLRLSSVTAADTAVYYCAKSMITTGFVMDSWGQGSLVTVSSGGGGSGGGGSGGGGSDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGAPTTTPAPRPPTPAPTIASQPLSLRPEASRPAAGGAVHTRGLDFAADPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 5 - protein, 533 aa, Artificial Sequence
  synthesized
MKHLWFFLLLVAAPRWVLSQVQLVQSGAEVKKPGASVKVSCKASGYTFSSSWMNWVRQAPGQRLEWMGRIYSGDGDAIYNGKFKGRVTITADTSASTAYMELSSLRSEDTAVYYCAREGKTGDLLLRSWGQGTLVTVSSGGGGSGGGGSGGGGSDIVMTQSPDSLAVSLGERATINCRASKSVSTSIYSYMHWYQQKPGQPPKLLIYYASYLESGVPDRFSGSGSGTDFTLTISSLQAEDVAVYYCEHSREFPFTFGQGTKLEIKGAPTTTPAPRPPTPAPTIASQPLSLRPEASRPAAGGAVHTRGLDFAADPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 6 - protein, 495 aa, Artificial Sequence
  synthesized
MKHLWFFLLLVAAPRWVLSEVQLVESGGGLVQPGGSLRLSCAASGFAFSIYDMSWVRQAPGKGLEWVSYISSGGGTTYYPDTVKGRFTISRDNAKNSLYLQMNSLRAEDTAVYYCARHSGYGSSYGVLFAYWGQGTLVTVSGGGGSGGGGSGGGGSDIQMTQSPSSLSASVGDRVTITCRASQDISNYLNWYQQKPGKAPKLLIYYTSILHSGVPSRFSGSGSGTDFTFTISSLQQEDIATYYCQQGNTLPWTFGGGTKVEIKGAPTTTPAPRPPTPAPTIASQPLSLRPEASRPAAGGAVHTRGLDFAADIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFRSKRSRGGHSDYMNMTPRRPGPTRKHYQPYAPPRDFAAYRSP

SEQ ID NO: 7 - protein, 581 aa, Artificial Sequence
  synthesized
MKHLWFFLLLVAAPRWVLSQVQLQESGPGLVKPSQTLSLTCTVSGVSLPDYGVSWIRQHPGKGLEWIGVIWGSETTYYNSALKSRVTISVDTSKNQFSLKLSSVTAADTAVYYCARHYYYGGSYAMDYWGQGTLVTVSSGGGGSGGGGSGGGGSDIQMTQSPSSLSASVGDRVTITCRASQDISKYLNWYQQKPGGAPKLLIYHTSRLHSGVPSRFSGSGSGTDFTFTISSLQQEDIATYYCQQGNTLPYTFGGGTKVEIKGAPTTTPAPRPPTPAPTIASQPLSLRPEASRPAAGGAVHTRGLDFAADWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKMKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 548 aa, Artificial Sequence
  synthesized
MKHLWFFLLLVAAPRWVLSQVQLQESGPGLVRPSQTLSLTCTVSGFTFTSYGVHWVRQPPGRGLEWIGVMWRGGSTDYNAAFMSRVTMLVDTSKNQFSLRLSSVTAADTAVYYCAKSMITTGFVMDSWGQGSLVTVSSGGGGSGGGGSGGGGSDIQMTQSPSSLSASVGDRVTITCKASEDIYNRLTWYQQKPGKAPKLLISGATSLETGVPSRFSGSGSGTDFTFTISSLQPEDIATYYCQQYWSNPYTFGQGTKVEIKGAPTTTPAPRPPTPAPTIASQPLSLRPEASRPAAGGAVHTRGLDFAADWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 9 - protein, 572 aa, Artificial Sequence
  synthesized
MEFGLSWVFLVALLRGVQCQVQLVQSGAEVKKPGASVKVSCKVSAYAFSSSWMNWVRQAPGKGLEWMGRIYPRDGDTNYNGKFKGRVTMTADTSTDTAYMELSSLRSEDTAVYYCAREGDGYYWYFDVWGQGTTVTVSSGGGGSGGGGSGGGGSEIVLTQSPATLSLSPGERATLSCRASQSIRNYLHWYQQKPGEAPRLLIYYASQSISGIPARFSGSGSGTDFTLTISSLETEDFAMYYCQHSNSWPLTFGQGTKLEIKGAPTTTPAPRPPTPAPTIASQPLSLRPEASRPAAGGAVHTRGLDFAADAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 10 - protein, 526 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKGAPPTTTPAPRPPTPAPTIASQPLSLRPEASRPAAGGAVHTRGLDFAADPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 11 - protein, 684 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMEFGLSWVFLVALLRGVQCYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 12 - protein, 670 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMKHLWFFLLLVAAPRWVLSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 13 - protein, 808 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMEFGLSWVFLVALLRGVQCYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDPAGGGTKTESSSRSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 14 - protein, 1119 aa, Artificial Sequence
  synthesized
YYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMEFGLSWVFLVALLRGVQCEVQLVESGGGLVQPGGSLRLSCAASGFTFSTYTMSWVRQTPAKGLVWVSTINSDGYNIYYSDSMKGRFTISRDNAKYTLYLQMNSLRAEDTAMYYCARCSYYSYDYFDYWGQGTLVTVSSGGGGSGGGGSGGGGSDIQMTQSPSSLSASVGDRVTITCRASENIDNYLAWYQQKQGKVPKLLIYAATNLADGMPSRFSGSGSGTDFTLTISSLQPEDVATYYCQHYYSNQLTFGQGTKLEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMEFGLSWVFLVALLRGVQCYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 15 - protein, 1024 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMEFGLSWVFLVALLRGVQCQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKPAGGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 16 - protein, 1195 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMEFGLSWVFLVALLRGVQCQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKPAGGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKPAGGGTKTESSSRGYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 17 - protein, 1301 aa, Artificial Sequence
  synthesized
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKGAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASGSGATNFSLLKQAGDVEENPGPMEFGLSWVFLVALLRGVQCQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKPAGGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKPAGGGTKTESSSRGYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDSGGGTKTESSSRGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 18 - protein, 716 aa, Artificial Sequence
  synthesized
MEFGLSWVFLVALLRGVQCQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKPAGGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKASGSGATNFSLLKQAGDVEENPGPMEFGLSWVFLVALLRGVQCYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 19 - protein, 349 aa, Artificial Sequence
  synthesized
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGSGGGSGAPTTTPAPRPPTPAPTIASQPLSLRPEACRPAAGGAVHTRGLDFACDIYIWAPLAGTCGVLLLSLVITLYCKKPAGRNTGPWLKKVLKCNTPDPSKFFSQLSSEHGGDVQKWLSSPFPSSSFSPGGLAPEISPLEVLERDKVTQLLLNTDAYLSLQELQGQDP

SEQ ID NO: 20 - protein, 818 aa, Artificial Sequence
  synthesized
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGSGGGSGAPTTTPAPRPPTPAPTIASQPLSLRPEACRPAAGGAVHTRGLDFACDIYIWAPLAGTCGVLLLSLVITLYCKKPAGRNTGPWLKKVLKCNTPDPSKFFSQLSSEHGGDVQKWLSSPFPSSSFSPGGLAPEISPLEVLERDKVTQLLLNTDAYLSLQELQGQDPASGSGATNFSLLKQAGDVEENPGPQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKPAGSGATNFSLLKQAGDVEENPGPMEFGLSWVFLVALLRGVQCYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 21 - protein, 940 aa, Artificial Sequence
  synthesized
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGSGGGSGAPTTTPAPRPPTPAPTIASQPLSLRPEACRPAAGGAVHTRGLDFACDIYIWAPLAGTCGVLLLSLVITLYCKKPAGRNTGPWLKKVLKCNTPDPSKFFSQLSSEHGGDVQKWLSSPFPSSSFSPGGLAPEISPLEVLERDKVTQLLLNTDAYLSLQELQGQDPASGSGATNFSLLKQAGDVEENPGPQVQLVESGGGVVQPGRSLRLSCAASGFTFSDYYMYWVRQAPGKGLEWVASISFDGTYTYYTDRVKGRFTISRDNSKNTLYLQMNSLRAEDTAVYYCARDRPAWFPYWGQGTLVTVSAGGGGSGGGGSGGGGSGDIVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPLTFGAGTKLELKPAGSGATNFSLLKQAGDVEENPGPMEFGLSWVFLVALLRGVQCYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDGGGTKTESSSRSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP